(19) United States
(12) Patent Application Publication
(10) Pub. No.: US 2024/0087756 A1
(43) Pub. Date: Mar. 14, 2024

(54) SYSTEMS AND METHODS FOR SUSCEPTIBILITY MODELLING AND SCREENING BASED ON MULTI-DOMAIN DATA ANALYSIS

(71) Applicant: Somalogic Operating Co., Inc., Boulder, CO (US)

(72) Inventors: Clare Paterson, Boulder, CO (US); Stephen Williams, Boulder, CO (US); Rachel Ostroff, Boulder, CO (US)

(21) Appl. No.: 17/932,223

(22) Filed: Sep. 14, 2022

(51) Int. Cl. G16H 50/70; G01N 33/574; G16B 50/20

(52) U.S. Cl. CPC G16H 50/70; G01N 33/57407; G16B 50/20

(57) ABSTRACT

Methods, systems, and computer-readable media for cancer susceptibility modelling and screening for a patient. The computer-readable medium includes executable instructions to perform a method for receiving input data associated with cancer and a patient, and determining a susceptibility model and a data enrichment rate based on the input data. The computer-readable medium also includes executable instructions for acquiring multi-domain patient data based on the susceptibility model and data enrichment rate, wherein the patient data comprises at least proteomic data. The computer-readable medium also includes executable instructions for generating cancer susceptibility data for the patient, and determining a cancer screening model for the patient based on the susceptibility data, using a machine learning approach. The computer-readable medium also includes executable instructions for screening the patient for cancer using the screening model, and iteratively refining the susceptibility model or the screening model based on one or more outcome metrics.

### BACKGROUND

In the fields of medical diagnostics and management, an ever-increasing amount of data and data sources are now available to researchers, analysts, organizational entities, and others. This influx of information allows for sophisticated analysis but, at the same time, presents many new challenges for sifting through the available data and data sources to locate the most relevant and useful information. As the use of technology continues to increase, so, too, will the availability of new data sources and information.

Because of the abundant availability of data from a vast number of data sources, determining the optimal values and sources for use presents a complicated problem difficult to overcome. Accurately and fully utilizing the available data across multiple sources can require both a team of individuals possessing extensive domain expertise as well as many months to years of work to evaluate the outcomes. The process can involve exhaustively searching through massive amounts of raw data to identify and study relevant data sources. Often, applying these types of analytical techniques to domains requiring accurate results obtainable only through time and resource intensive research is incompatible with modern applications' demands. For example, the developed process for evaluating outcomes may not line up with specific circumstances or individual considerations. In this scenario, applying the process requires extrapolation to fit the specific circumstances, to dilute the process's effectiveness, or to require spending valuable time and resources to modify the process. As a result, processes developed in this way typically provide only generalized guidance insufficient for repurposing in other settings or by other users. As more detailed and individualized data becomes available, demand for the ability to accurately discern relevant data points from the sea of available information across multiple data sources, and efficiently apply that data across myriads of personalized scenarios increases.

Multi-domain data processing and analysis play a crucial role in the diagnostic and management of diseases such as cancer, which is a complex, heterogeneous disease modulated by multiple factors across many domains, including genetic, molecular, cellular, tissue, population, environmental, and socioeconomic factors. Early diagnosis of cancer, for example, is instrumental in improving outcomes by providing care at the earliest possible stage and may provide multiple benefits to the patient, including less aggressive treatments, improved quality-of-life, and overall survival rate. As a result, public health programs place a special emphasis on effective screening regimen for early cancer detection.

### SUMMARY

Certain embodiments of the present disclosure relate to a non-transitory computer readable medium including instructions that are executable by one or more processors to cause a system to perform a method for cancer susceptibility-modelling and screening. The method may include receiving input data associated with one or more cancers and a patient; determining a susceptibility model and a data enrichment rate based on the input data; acquiring data associated with the patient from a plurality of data domains based on the susceptibility model and the data enrichment rate, wherein the patient data comprises at least proteomic data. The method may also include generating, using one or more machine learning algorithms, a set of susceptibility data associated with the patient based on the susceptibility model and the patient data. The method may also include determining, using one or more machine learning algorithms, a screening model for the patient based the susceptibility data, and screening the patient for the one or more types of cancer based on the screening model.

According to some disclosed embodiments, the input data may comprise at least a cancer type, a cancer prevalence, a cancer prognosis, a cancer stage, timing of cancer diagnosis, or any combination thereof.

According to some disclosed embodiments, the patient data may further comprise patient characteristics data, medical history data, genetic data, immunological data, insurance data, healthcare coverage data, environmental data, or biological sampling data from the patient.

According to some disclosed embodiments, the proteomic data may be based on a biological sample of the patient.

According to some disclosed embodiments, the method may further comprises determining, from the patient data, a set of features associated with the patient's susceptibility to the cancer.

According to some disclosed embodiments, determining a susceptibility model may comprise selecting a susceptibility model from a plurality of data models within a model databank.

According to some disclosed embodiments, the screening model of the cancer for the patient may comprise one or more screening methods, each associated with one or more screening schedules.

According to some disclosed embodiments, the method may further comprise iteratively refining the screening model using one or more machine learning algorithms, by adjusting a screening schedule of a screening method based on one or more outcome metrics, until the one or more outcome metrics reach a threshold value.

According to some disclosed embodiments, the outcome metric may comprise a positive predictive value, a screening burden measurement, an estimated risk measurement, or any combination thereof.

According to some disclosed embodiments, the method may further comprise iteratively refining the susceptibility model using one or more machine learning algorithms, by adjusting the data enrichment rate based on the one or more outcome metrics and generating a refined set of susceptibility data based on the adjusted enrichment rate; until the one or more outcome metrics reach a threshold value.

Other systems, methods, and computer-readable media are also discussed within.

### BRIEF DESCRIPTION OF THE DRAWINGS

The accompanying drawings, which are incorporated in and constitute a part of this specification, illustrate several embodiments and, together with the description, serve to explain the disclosed principles. In the drawings:

FIG. 1 is a block diagram illustrating an example system for determining and refining susceptibility models and screening models based on data input from multiple data sources, according to some embodiments of the present disclosure.

FIG. 2 is a block diagram illustrating an example susceptibility model engine for generating and refining susceptibility data based on multi-domain data, according to some embodiments of the present disclosure.

FIG. 3 is a block diagram illustrating an example screening model engine for screening modulation and predictive output generation, validation, and refinement, according to some embodiments of the present disclosure.

FIG. 4 is a block diagram illustrating an example machine learning platform, according to some embodiments of the present disclosure.

FIG. 5 illustrates a schematic diagram of an example server of a distributed system, according to some embodiments of the present disclosure.

FIG. 6 is a flow diagram illustrating an example process for receiving input based on a potential outcome, performing multi-domain data acquisition, generating individualized data, performing screening and refinement of the data and model based on measured performance, according to some embodiments of the present disclosure.

### DETAILED DESCRIPTION

In the following detailed description, numerous specific details are set forth in order to provide a thorough understanding of the disclosed example embodiments. However, it will be understood by those skilled in the art that the principles of the example embodiments may be practiced without every specific detail. Well-known methods, procedures, and components have not been described in detail so as not to obscure the principles of the example embodiments. Unless explicitly stated, the example methods and processes described herein are neither constrained to a particular order or sequence nor constrained to a particular system configuration. Additionally, some of the described embodiments or elements thereof can occur or be performed simultaneously, at the same point in time, or concurrently. Reference will now be made in detail to the disclosed embodiments, examples of which are illustrated in the accompanying drawings.

In view of the emerging paradigm for personalized cancer screening and diagnosis based on each patient's individual susceptibility to one or more specific cancer types, traditional analytical approaches that focus solely on single-factor analysis (e.g., isolated genetic studies) may prove to be of limited value. The utilization of the patient's comprehensive proteomic data (e.g., by harnessing the immune response of myriads of proteins in the form of seropositivity to tumor antigens) has the potential to complement these types of approaches for cancer detection. Moreover, individualized modelling of cancer susceptibility may be achieved by analyzing proteomic patterns in synergistic combination with data from multiple other domains, such as patient characteristics data and environmental data. This type of data enrichment can help to further tailor the susceptibility modelling for each individual patient with respect to a specific type of cancer and may effectively compensate for rare cancer types of low prevalence in a given population, which are often less detectable and more difficult to screen.

Effective cancer susceptibility modelling would in turn enable an optimization of the patient's cancer screening regimen. For instance, traditional screening regimen, without taking into account the patient's individual susceptibility, are often associated with low positive predictive values in the cancer diagnosis, leading to a high false-positive rate of diagnosis. This in turn requires additional confirmatory testing with expensive imaging studies (e.g., CT, MRI, PET scans etc.) or invasive techniques such as tissue biopsy, thus incurring the cost of unnecessary screening and potentially inflicting extraneous harm to the patient, all the while without reaching a reliable diagnosis. At the same time, the screening schedule (e.g., the frequency of administering a specific screening method to the patient) may be inappropriately modulated without consideration of the patient's specific susceptibility to the disease. For instance, some screening techniques (e.g., colonoscopy, fine-needle aspiration biopsies) may not be cost-effective or could even be harmful to be administered at a high-level interval to patients of a certain level of disease susceptibility if they only result in an inconsequential amount of lead-time in diagnosis (e.g., without having any impact on disease management).

In this regard, artificial intelligence systems and machine learning algorithms may be an invaluable tool for building a comprehensive yet individualized screening model for a given patient that is based on the patient's susceptibility data. For instance, a screening model based on a machine-learning approach could assist in identifying high-risk patients with regard to a particular type of cancer according to the patient's individual susceptibility and would allow for calibration of the screening regimen based on susceptibility and risk stratification. For instance, an exemplary system may increase the screening frequency for higher-risk patients or decrease the frequency for lower-risk patients. An exemplary system may also change the screening modality (e.g., deploying a higher-cost, more invasive procedure such as a colonoscopy for patients at higher risk for colorectal cancer, or using a lower-cost, less invasive procedure such as fecal occult blood testing for low-risk patients. In addition, a machine-learning based screening model would also be capable of maintaining a high level of robustness through iterative refinement based on one or more performance measurements (e.g., based on its predictive accuracy or the burden of the screening) in order to determine the most suitable screening schedule and the most reliable diagnosis, while also having the capacity to output additional patient-specific recommendations such as lifestyle modifications etc.

The embodiments described herein provide technologies and techniques for evaluating large numbers of data sources and vast amounts of data used in the creation of a machine learning model. These technologies can use information relevant to the specific domain and application of a machine learning model to prioritize potential data sources. Further, the technologies and techniques herein can interpret the available data sources and data to extract probabilities and outcomes associated with the machine learning model's specific domain and application. The described technologies can synthesize the data into a coherent machine learning model, which can be used to analyze and compare various paths or courses of action.

These technologies can efficiently evaluate data sources and data, prioritize their importance based on domain and circumstance specific needs, and provide effective and accurate predictions that can be used to evaluate potential courses of action. The technologies and methods allow for the application of data models to personalized circumstances. These methods and technologies allow for detailed evaluation that can improve decision making on a case-by-case basis. Further, these technologies can evaluate a system where the process for evaluating outcomes of data may be set up easily and repurposed by other uses of the technologies.

Technologies may utilize machine learning models to automate the process and predict responses without human intervention. The performance of such machine learning models is usually improved by providing more training data. A machine learning model's prediction quality is evaluated manually to determine if the machine learning model needs further training. Embodiments of these technologies described can help improve machine learning model predictions using the quality metrics of predictions requested by a user.

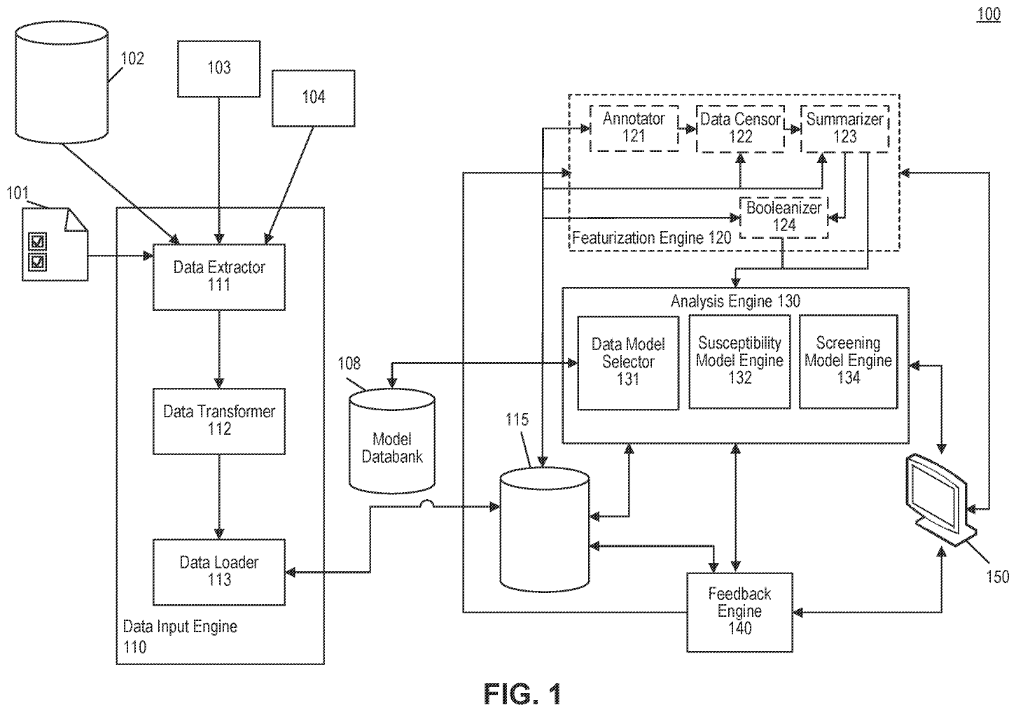

FIG. 1 is a block diagram illustrating various exemplary components of a system 100 for determining and refining susceptibility models and screening models based on data input from multiple data sources, consistent with embodiments of the present disclosure. System 100 can include data input engine 110 that can further include data extractor 111, data transformer 112, and data loader 113. Data input engine 110 can process data from data sources 101-104. In some embodiments, data input engine 110 can be implemented using a computing device. For example, data from data sources 101-104 can be obtained through I/O devices or network interfaces. Further, the data can be stored during processing in a suitable storage or system memory. Data input engine 110 can also interact with data storage 115. Data storage 115 can further be implemented on a computing device that stores data in storage or system memory. System 100 may include featurization engine 120. Featurization engine 120 may comprise annotator 121, data censor 122, summarizer 123, and booleanizer 124. System 100 may also include analysis engine 130 and feedback engine 140. Similar to data input engine 110, featurization engine 120 can be implemented on a computing device. Similarly, featurization engine 120 can utilize storage or system memory for storing data and can utilize one or more I/O devices or network interfaces for transmitting or receiving data. Each of data input engine 110, data extractor 111, data transformer 112, data loader 113, featurization engine 120, annotator 121, data censor 122, summarizer 123, booleanizer 124, analysis engine 130, and feedback engine 140 can be a module, which is a packaged functional hardware unit designed for use with other components or a part of a program that performs a particular function of related functions. Each of these modules can be implemented using a computing device. Each of these components is described in more detail below. In some embodiments, the functionality of system 100 can be split across multiple computing devices to allow for distributed processing of the data. In these embodiments, the different components can communicate over one or more I/O devices or network interfaces.

System 100 can be related to many different domains or fields of use. Descriptions of embodiments related to specific domains, such as disease diagnosis and management (e.g., relating to cancer), is not intended to limit the disclosed embodiments to those specific domains, and embodiments consistent with the present disclosure can apply to any domain that utilizes predictive modeling based on available data.

Data input engine 110 is a module that can retrieve data from a variety of data sources (e.g., data source 101, 102, 103, and 104) and process the data so that it can be used with the remainder of system 100. Data input engine 110 can further include data extractor 111, data transformer 112, and data loader 113.

Data extractor 111 retrieves data from data sources 101, 102, 103, and 104. Each of these data sources can represent a different type of data source. For example, data source 101 can be a database. Data source 102 can represent structured data. Data sources 103 and 104 can be flat files. Further, data sources 101-104 can contain overlapping or completely disparate data sets. In some embodiments, data sources 101-104 may comprise input data associated with a potential outcome. For instance, in the cancer screening or diagnosis setting, input data may comprise data associated with a cancer type, a cancer prevalence, a cancer prognosis, timing of cancer diagnosis, or a cancer stage. In some embodiments, data sources 101-104 may comprise a value comprising a data enrichment rate. In some embodiments, the data enrichment rate value is based on user input. In some embodiments, data source 101 can contain proteomic data while data sources 102, 103, and 104 contain various data from other domains or sources. For instance, data source 102 may contain patient characteristics data such as the age, gender, race/ethnicity, height/weight of the patient. In another example, data source 103 may contain environmental data, comprising smoking history, diet type, etc. In another example, data source 104 may contain data obtained from a biological sampling from the patient, such as data relating to a sample of blood, plasma, serum, or urine. In another example, data source 101-104 may comprise demographical data, medical history data, clinical visit data, or surgical history data. In another example, data sources 101-104 may comprise family history data. In another example, data sources 101-104 may comprise genetic data or immunological data. In another example, data sources 101-104 may comprise insurance data or healthcare coverage data. Data extractor 111 can interact with the various data sources, retrieve the relevant data, and provide that data to data transformer 112.

Data transformer 112 can receive data from data extractor 111 and process the data into standard formats. In some embodiments, data transformer 112 can normalize data such as dates or numerical values based on specific units of measure. For example, data source 101 can store dates in day-month-year format, while data source 102 can store dates in year-month-day format, or while data source 103 can store body weights as measured in kilograms or pounds. In this example, data transformer 112 can modify the data provided through data extractor 111 into a consistent date format or standardized unit format, respectively. Accordingly, data transformer 112 can effectively clean the data provided through data extractor 111 so that all of the data, although originating from a variety of sources, has a consistent format.

Moreover, data transformer 112 can extract additional data points from the data. For example, data transformer can process a date in year-month-day format by extracting separate data fields for the year, the month, and the day. Data transformer can also perform other linear and non-linear transformations and extractions on categorical and numerical data such as normalization and demeaning. Data transformer 112 can provide the transformed or extracted data to data loader 113.

Data loader 113 can receive the normalized data from data transformer 112. Data loader 113 can merge the data into varying formats depending on the specific requirements of system 100 and store the data in an appropriate storage mechanism such as data storage 115. In some embodiments, data storage 115 can be data storage for a distributed data processing system (e.g., Hadoop Distributed File System, Google File System, ClusterFS, or OneFS). In some embodiments, data storage 115 can be a relational database (described in more detail below). Depending on the specific embodiment, data loader 113 can optimize the data for storing and processing in data storage 115. In some embodiments, various types of data structures, such as a database, a flat file, data stored in memory (e.g., system memory), or data stored in any other suitable storage mechanism can be stored by data loader 113 in data storage 115.

System 100 may optionally include featurization engine 120. Featurization 120 can process the data prepared by data input engine 110 and stored in data storage 115. Featurization engine 120 can include annotator 121, data censor 122, summarizer 123, and booleanizer 124. Featurization can retrieve data from data storage 115 that has been prepared by date input engine 110. For example, various types of data structures, such as a database, a flat file, data stored in memory (e.g., system memory), or data stored in any other suitable storage mechanism which can be stored by data loader 113 in data storage 115, can be suitable inputs to featurization engine 120.

Featurization engine 120 can convert the data into features that can then be used for additional analysis. A feature can be data that is representative of other data. Features can be determined based on the domain, data type of a category, or many other factors associated with data stored in a data structure. Additionally, a feature can represent information about multiple data records in a data set or information about a single category in a data record. Moreover, multiple features can be produced to represent the same data.

Featurization engine 120 can contain annotator 121. Annotator 121 can provide context to the data structures from data storage 115. Annotator 121 can further determine which additional data records are associated with a target event and should be used in a predictive model. After annotator 121 processes and identifies relevant limits on the data, data censor 122 can filter out data that does not meet an established criterion. After the data has been censored, summarizer 123 can analyze remaining data structures and data to produce features for the data set. In some embodiments, features can be based on the specific type of data under consideration, and many features can be produced from a single data point or set of data points. Summarizer 123 can further consider data points occurring across multiple data records for an individual, or can consider data points related to multiple individuals.

After features have been established for a particular data set, the established features can be stored in data storage 115, provided directly to analysis engine 130, or provided to booleanizer 124 for additional processing before analysis.

Booleanizer 124 can process the determined features from summarizer 123 and establish corresponding boolean or binary data for those features. Using a binary representation of the features can allow the data set to be analyzed using statistical analysis techniques optimized for binary data. Booleanizer 124 can produce boolean or binary values based on whether or not a specific feature or attributes exists. For example, a feature of the data that establishes whether or not a particular type of claim exists for a user can easily be represented by a “1” for “True” and a “0” for “False.” In this example, the feature can be whether or not an individual has a specific protein level above a certain numerical threshold, or whether the individual is a smoker, or has a family history of cancer.

After processing the data, featurization engine 120 can produce feature data directly from summarizer 123 or binary feature data from booleanizer 124. This data can be stored in data storage 115 for later analysis or passed directly to analysis engine 130.

Analysis engine 130 may analyze data stored by data loader 113 or in data storage 115. In some embodiments, Analysis engine 130 may analyze normalized data based on multiple data sources, as exemplified by data sources 101-104. Analysis engine 130 may include data model selector 131, susceptibility model engine 132, or screening model engine 134.

Data model selector 131 may select a data model from model databank 108. A selected data model may be a type of susceptibility model or a type of screening model. A selected model may be based on the data obtained from data input engine 110, or data storage 115. In some embodiments, data model selector 131 may select a data model based on input data from data input engine 110 or data storage 115, or based on information requested by user interface 150. In some embodiments, exemplary input data may comprise data associated with a type of cancer or a prevalence of the cancer. In some embodiments, exemplary input data may comprise data associated data associated with patients such as age, gender, race/ethnicity, history of smoking, or personal or family history of cancer.

In some embodiments, data model selector 131 may transmit a selected data model to susceptibility model engine 132. Susceptibility model engine 132 may receive a data model from data model selector 131 or from model databank 108. Susceptibility model engine 132 may receive data from data stored by data loader 113 or in data storage 115. In some embodiments, susceptibility model engine 132 may receive normalized data based on multiple data sources, as exemplified by data sources 101-104. Susceptibility model engine 132 may receive a value comprising a data enrichment rate from data input engine 110 or data storage 115. In a cancer screening or diagnosis setting, susceptibility model engine 132 may also receive input data associated with a cancer type, a cancer prevalence, a cancer prognosis, timing of cancer diagnosis, or a cancer stage. Susceptibility model engine 132 may also receive input data. In some embodiments, susceptibility model engine 132 may perform data enrichment of the received data. In some embodiments, susceptibility model engine 132 may optionally perform feature selection based on the receive data. Susceptibility model engine 132 may also apply received data to the selected model. Susceptibility model engine 132 may also estimate susceptibility. Susceptibility model engine 132 may also generate susceptibility data based on the estimated susceptibility. In some embodiments, susceptibility model engine 132 may also validate the generated data based on input from feedback engine 140. In some embodiments, susceptibility model engine 132 may also refine generated data based on data validation input. In some embodiments, data refinement is performed by adjusting the data enrichment rate. In some embodiments, the data validation input is based on feedback engine 140. In some embodiments, susceptibility model engine 132 may transmit susceptibility data to screening model engine 134.

In some embodiments, data model selector 131 may transmit a selected data model to screening model engine 134. Screening model engine 134 may receive a data model from data model selector 131. Screening model engine 134 may receive data from data stored by data loader 113 or in data storage 115. In some embodiments, screening model engine 134 may receive normalized data based on multiple data sources, as exemplified by data sources 101-104. Screening model engine 134 may receive susceptibility data from susceptibility model engine 132. In some embodiments, screening model engine 134 may select a screening method. Screening model engine 134 may also perform modulation of a screening frequency for the selected screening method. In some embodiments, screening model engine 134 may also apply a screening method at a modulated screening frequency to generate a predictive output value. Screening model engine 134 may also validate the predictive output value based on input from feedback engine 140. In some embodiments, susceptibility model engine 134 may also perform refinement of screening. In some embodiments, a screening refinement is performed by selecting a new screening method or modulating a screening frequency.

Analysis engine 130 may optionally analyze the features or binary data produced by featurization engine 120 to determine which features are most indicative of the occurrence of the target event. Analysis engine 130 can use a variety of methods for analyzing the many thousands, millions, or billions of features that can be produced by featurization engine 120. Examples of analysis techniques include feature subset selection, stepwise regression testing, chi-squared (χ2) testing, or other regularization methods that encourage sparsity (e.g., coefficient shrinkage). Analysis engine 130 can use this output to produce a model for application to existing and future data to identify individuals who will likely experience the target event (e.g., a positive diagnosis of a cancer type).

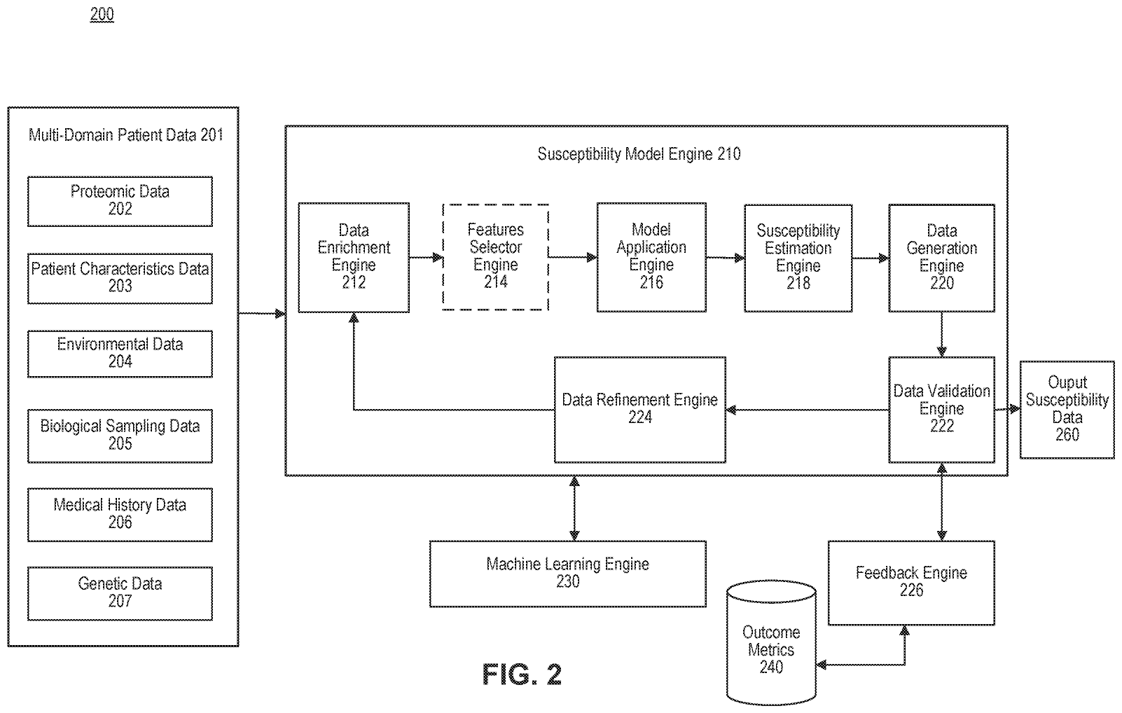

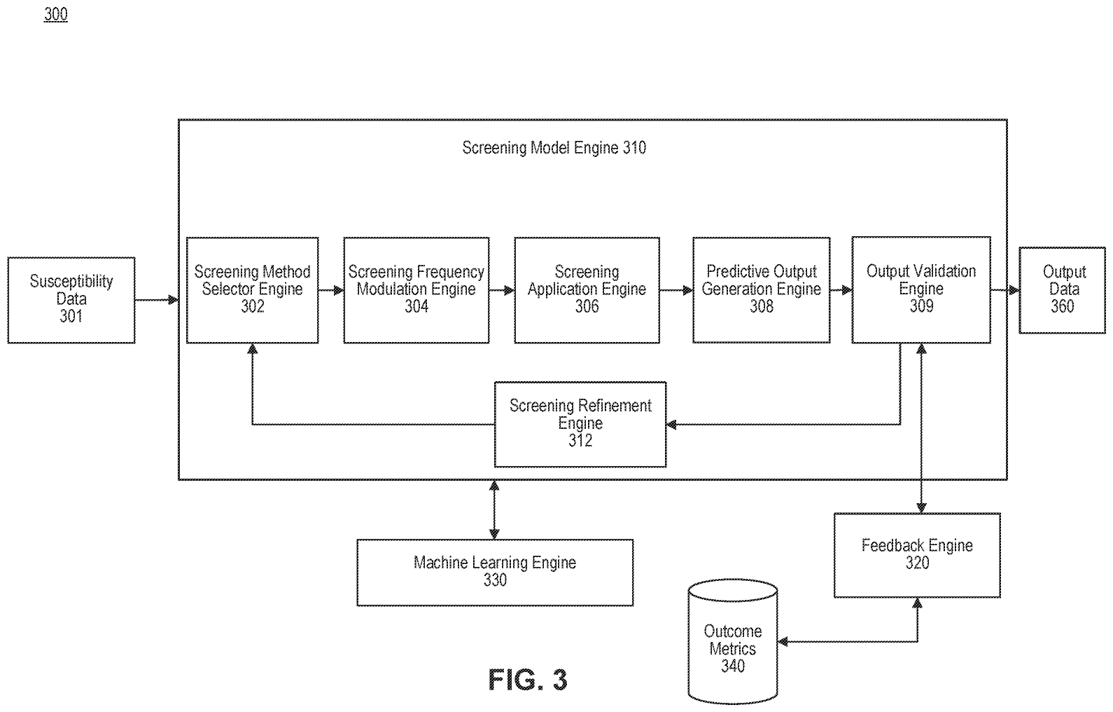

Analysis engine 130 can store data models in data storage 115 for future use. Additionally, the data model can be provided to feedback engine 140 for refinement. Feedback engine 140 can apply the data model to a wider set of data to determine the accuracy of the model. Feedback engine 140 can utilize one or more outcome metrics as in 240 in FIGS. 2 and 340 in FIG. 3 to measure the performance of the model. Based on those results, feedback engine 140 can report results back to analysis engine 130. Feedback engine 140 can report back to susceptibility model engine 132 to refine the generated susceptibility data (as shown in FIG. 2). Feedback engine 140 can also report back to screening model engine 134 to refine the screening model (as shown in FIG. 3). In some embodiments, feedback engine 140 can optionally report back to featurization engine 120 to iteratively update the specific inputs used by annotator 121, data censor 122, and summarizer 123 to adjust the model. In this way, featurization engine 120 can be trained as more and more data is analyzed.

In some embodiments, analysis engine 130 can use a variety of statistical analysis techniques to test the accuracy and usefulness of a specific model or of multiple models generated for a target event. The models can be evaluated using evaluation metrics such as, among others, precision, recall, accuracy, area under the receiver operator characteristic (ROC) curve, the area under the precision recall (PR) Curve, lift, or precision at rank. Feedback engine 140 can provide feedback that is intended to optimize the model based on the specific domain and use case for the model. For example, in a cancer diagnosis context, if the model is being used to identify individuals with high susceptibility to a specific cancer type, feedback engine 140 can provide feedback and adjustments to data model selector 131, susceptibility model engine 132, screening model engine 134, or optionally featurization engine 220, to optimize the model for higher precision in order to ensure accuracy of diagnosis by minimizing false positives, with the understanding that false positives could lead to unwarranted additional testing which may be costly or invasive. Additionally, feedback engine 140 can test a data model using techniques such as cross validation in order to optimize for the number of features chosen for the model by analysis engine 130.

System 100 can further include user interface 150. User interface 150 can be a graphical user interface (GUI) implemented on a computing device, utilizing a graphic memory, GPU(S), and a display device. User interface 150 can provide a representation of the data from, analysis engine 130 or feedback engine 140, or optionally featurization engine 120. User interface 150 can be a read-only interface that does not accept user input. In some embodiments, user interface 150 can accept user input to control the representation. In other embodiments, user interface 150 can accept user input to control or modify the components of system 100. User interface 150 can be text based or can include graphical components that represent the displayed data.

In some embodiments, user interface 150 can be provided to a user in order to make recommendations based on the predictive model generated by system 100. For example, system 100 can be used to generate a predictive model for diagnosis of a specific cancer type. The results of this model can be presented to patients whose data can indicate that they possess a certain level of susceptibility to that specific cancer type. The individual users will have no insight into the specific data model, itself, but will benefit from the ability to seek preventive care based on the diagnosis.

In some embodiments, user interface 150 can provide a representation of the functioning of featurization engine 120, analysis engine 130, or feedback engine 140. In some embodiments, user interface 150 can display feedback information from feedback engine 140. In these embodiments, domain experts can use user interface 150 to verify the generated models, provide feedback regarding the generated models, and or modify the inputs or data used by analysis engine 130 or featurization engine 120 to generate the models.

System 100 can be used as described to quickly and accurately produce effective predictive models across many different domains. Instead of requiring labor and time intensive methods for generating narrow predictive models, system 100 can be used to quickly generate and iterate on predictive models that are generic enough to be applied to wide ranges of future data while at the same time utilize statistically significant features to best predict a target event.

FIG. 2 is a block diagram illustrating an exemplary susceptibility model engine 210 for generating and refining susceptibility data based on multi-domain patient data 201, according to some embodiments of the present disclosure.

Susceptibility model engine 210 may include data enrichment engine 212, model application engine 216, susceptibility estimation engine 218, data generation engine 220, data validation engine 222, and data refinement engine 224. Susceptibility model engine 210 may optionally include features selector engine 214. In some embodiments, susceptibility model engine 210 may be exemplified by susceptibility model engine 132 as shown in FIG. 1.

Multi-domain patient data 201 may include proteomic data 202, patient characteristic data 203, environmental data 204, biological sampling data 205, medical history data 206, or genetic data 207. In some embodiments, multi-domain patient data 201 may be exemplified by data stored by data loader 113 or in data storage 115. In some embodiments, multi-domain patient data 201 may be exemplified by normalized data based on multiple data sources, such as data sources 101-104 in FIG. 1.

Susceptibility model engine 210 may receive multi-domain patient data 201 from multiple data sources as input. In some embodiments, the multiple data sources may include, but are not limited to, data from multiple domains or modalities. For instance, in a disease diagnosis setting (e.g., for cancer detection), the data sources may comprise, but are not limited to, proteomic data 202, patient characteristics data 203, environmental data 204, biological sampling data 205, medical history data 206, family history data, genetic data 207, or immunological data. The data sources may also comprise insurance data or healthcare coverage data.

Proteomic data may comprise data associated with one or more set of proteins obtained from a patient's biological sampling. In some embodiments, biological sampling may comprise obtaining a blood, fluid, or tissue sample from a patient. Proteomic data may comprise the individual abundance values of a set of proteins obtained from biological sampling or the individual immune responses of a set of proteins obtained from biological sampling in the form of seropositivity to one or more tumor antigens. In some embodiments, proteomic data may comprise mass spectrometry data, data associated with identified peptides, post-translational modifications data; fluorescence microscopy data, protein subcellular localizations data, fluorescence energy transfer experimental data, protein domain and three-dimensional structure predictions data, protein-protein interactions data, or protein-protein interaction prediction data, associated with one or set of proteins obtained from biological sampling.

In some embodiments, patient characteristics data may comprise data associated with age, gender, race/ethnicity, height/weight of the patient. In some embodiments, patient characteristics data may further comprise demographical data. In some embodiments, environmental data may comprise data associated with smoking history or diet type (e.g., a predominantly red-meat diet or an all-vegetarian diet). In some embodiments, biological sampling data may comprise data relating to a sample of blood, plasma, serum, or urine obtained from a patient. In some embodiments, medical history data may comprise clinical visit data or surgical history data. In some embodiments, family history data may comprise hereditary history of positive cancer diagnoses or treatments associated with family members of a patient. In some embodiments, genetic data may comprise data associated with tumor-related genetic markers for a patient. In some embodiments, immunological data may comprise data associated with immune or autoimmune responses related to one or more tumor markers, or individualized response to immunotherapy.

In some embodiments, multi-domain patient data 201 may be processed, transformed, normalized, or stored in data storage by a data input engine. In some embodiments, a data input engine may be exemplified by data input engine 110 in FIG. 1. In some embodiments, a featurization engine may extract a set of data features from multi-domain patient data 201. In some embodiments, a featurization engine may be exemplified by featurization engine 120 as in FIG. 1.

Susceptibility model engine 210 may include data enrichment engine 212. Data enrichment engine 212 may receive a value comprising a data enrichment rate from data input engine 110 or data storage 115. In some embodiments, the data enrichment rate value may comprise a coefficient (e.g., 2×, 5×, 10×, etc.) or a percentage (e.g., 10%, 20%, etc.). Data enrichment engine 212 may enrich the multi-domain patient data 201 based on the data enrichment rate. For instance, based on a data enrichment rate of 10%, data enrichment engine 212 may increase the amount of data received from data input engine 110 or data storage 115 accordingly by 10%. In another example, data enrichment engine 212 may double the amount of data received based on a data enrichment rate of 2×.

Susceptibility model engine 210 may optionally include features selector engine 214. Features selector engine 214 may receive a set of features from featurization engine 120, as in FIG. 1. In some embodiments, the set of features may be based on input data from data input engine 110, data from data storage 115 (as shown in FIG. 1). In some embodiments, the set of features may be based on multi-domain patient data 201.

Susceptibility model engine 210 may include model application engine 216. In some embodiments, model application engine 216 may receive a selected data model from data model selector 130 and analysis engine 130, as shown in FIG. 1. Model application engine 216 may apply a subset of multi-domain patient data 201 to the selected data model by selecting data which fits into one or more data parameters of the selected data model. In some embodiments, the selected subset of multi-domain patient data 201 may be enriched by data enrichment engine 212. For instance, model application engine 216 may select a data model that is based on proteomic data or environmental data. Model application engine 216 may select a subset of multi-domain patient data 201 comprising proteomic data 202 or environmental data 204. Data enrichment engine 212 may enrich the selected data based on the data enrichment rate. Model application engine 216 may apply the data to corresponding data parameters in the selected data model. In some embodiments, model application engine 216 may optionally apply a set of features received from features selector engine 214 to data parameters within the selected model.

Susceptibility model engine 210 may include susceptibility estimation engine 218. Susceptibility estimation engine 218 may calculate a susceptibility score based on a selected data model or an application of data to the selected model from model application engine 216. In some embodiments, for instance in the cancer diagnosis setting, a susceptibility score may comprise a numerical percentage representing a patient's likelihood of having a specific type of cancer. In some embodiments, a susceptibility score may comprise a category representing a patient's level of risk stratification with regard to a specific type of cancer (e.g., high-risk, intermediate risk, or low risk). In some embodiments, a susceptibility score may comprise a multiplicative coefficient representing a patient's risk of developing a specific cancer type relative to a reference population (e.g., 2× or 10× risk of developing a cancer).

Susceptibility model engine 210 may include data generation engine 220. Data generation engine 220 may generate a set of susceptibility data based on a data model and an application of data (e.g., a subset of the multi-domain patient data 201) to the data model from model application engine 216. Data generation engine 220 may generate a set of susceptibility data based on a susceptibility score from susceptibility estimation engine 218. In the cancer diagnosis setting, data generation engine 220 may generate a set of susceptibility data for an individual patient with regard to a specific cancer type. The generated susceptibility data may comprise a susceptibility score from susceptibility estimation engine 218, or additional data associated with susceptibility estimation as performed by engine 218. For instance, the generated susceptibility data may comprise a susceptibility score (e.g., in the form of a percentage or category of risk stratification), a subset of the multi-domain patient data 201 (e.g., proteomic data 202, environmental data 204), or data parameters from a selected data model from data model selector 131 (as in FIG. 1).

Susceptibility model engine 210 may include data validation engine 222. Data validation engine 222 may receive susceptibility data generated by data generation engine 220. In some embodiments, data validation engine 222 may perform outputting of the generated susceptibility data, as exemplified by output susceptibility data 260. In some embodiments, output susceptibility data 260 may be stored in a database, as exemplified by data storage 115 or data sources 101-104 as shown in FIG. 1. Data validation engine 222 may perform validation of the generated susceptibility data by interacting with feedback engine 226. In some embodiments, feedback engine 226 may be exemplified by feedback engine 140 in FIG. 1. In some embodiments, feedback engine 226 may apply the selected data model to a wider set of data to determine the accuracy of the model. In some embodiments, feedback engine 226 may utilize one or more outcome metrics as in outcome metrics 240 and generate a set of performance measurement data based on the generated susceptibility data or the selected data model. In some embodiments, outcome metrics 240 may comprise a positive predictive value, a screening burden measurement, or an estimated risk measurement. For instance, in the cancer diagnosis setting, outcome metrics 240 may comprise a positive predictive value as an indication of the rate of accurate predictive output if cancer diagnosis by predictive output generation engine 308 in screening model engine 310 (as shown in FIG. 3). A screening burden measurement may be a measure based on a cost-effectiveness analysis of applying a cancer screening method at a specific screening frequency by screening application engine 306 in screening model engine 310 (as shown in FIG. 3). A screening burden may be a measure of cost-effectiveness based on the patient's insurance data or healthcare coverage data. An estimated risk measurement may be a measure based on any reported harm (e.g., from invasive screening techniques such as needle biopsies) to a patient due to the application of a screening method at a specific screening frequency. In some embodiments, the set of performance measurement data may comprise a numerical analysis based on a positive predictive value from outcome metrics 240. In some embodiments, the set of performance measurement data may comprise a numerical cost-effectiveness analysis of applying a screening method (e.g., a cancer screening method in the cancer diagnosis setting) at a given screening frequency. In some embodiments, the set of performance measurement data may comprise an estimated risk analysis based on reported harm (e.g., from invasive screening techniques to a patient in the cancer diagnosis setting) due to the application of the screening method at a given screening frequency.

Feedback engine 226 may transmit the performance measurement data to data validation engine 222. Data validation engine 222 may generate a data validation score for data refinement based on the performance measurement data. In some embodiments, the data validation score for data refinement may comprise a binary value (e.g., “data refinement needed”, “data refinement not needed”) or a gradient of values on a numerical scale (e.g., on a scale of 1-10, the need for data refinement with regard to a specific data set is a “6 out of 10”). Data validation engine 222 may transmit the data validation score for data refinement to data refinement engine 224.

Susceptibility model engine 210 may include data refinement engine 224. Data refinement engine 224 may receive a data validation score for data refinement from data validation engine 222 or performance measurement data from feedback engine 226. Data refinement engine 224 may perform data refinement based on input from data validation engine 222 or a performance measurement data from feedback engine 226. Data refinement engine 224 may perform data refinement by calculating an adjusted data enrichment rate based on the performance measurement data. Data refinement engine 224 may transmit the adjusted data enrichment rate to data enrichment engine 212 for iterative data refinement.

In some embodiments, susceptibility model engine 210 may enable iterative cycles of data refinement. An iterative cycle of data refinement may comprise data refinement engine 224 transmitting an adjusted data enrichment rate to data enrichment engine 212. An iterative cycle may comprise data enrichment engine 212 performing data enrichment based on the adjusted data enrichment rate. For instance, if an adjusted data enrichment rate is determined to be 20% by data refinement engine 224 based on the output of data validation engine 222 or feedback engine 226, and the original unadjusted data enrichment rate was 10%, then data enrichment engine 212 may increase its rate of data enrichment by 10% to generate a refined data set. In some embodiments, the refined dataset is a subset of multi-domain patient data 201 calibrated by the adjusted data enrichment rate. An iterative cycle of data refinement may optionally comprise features selector engine 214 selecting a set of features based on the refined dataset. An iterative cycle of data refinement may also comprise model application engine 216 applying the refined dataset to a selected data model. An iterative cycle of data refinement may also comprise susceptibility estimation engine 218 performing susceptibility estimation based on the refined dataset and data model. An iterative cycle of data refinement may also comprise data generation engine 220 generating a set of refined susceptibility data based on the output from susceptibility estimation engine 218. An iterative cycle of data refinement may also comprise data validation engine 222 interacting with feedback engine 226 to validate the refined susceptibility data. In some embodiments, iterative cycles of data refinement may continue until data validation engine 222 or feedback engine 226 determines that one or more outcome metrics 240 has achieved a threshold value. In some embodiments, data validation engine 222 may perform outputting of the set of refined susceptibility data, as exemplified by output susceptibility data 260. In some embodiments, output susceptibility data 260 may be stored in a database, as exemplified by data storage 115 or data sources 101-104 as shown in FIG. 1.

Susceptibility model engine 210 may interact with machine learning engine 230. According to some embodiments, machine learning engine 230 may analyze one or more dataset or data subsets utilized by susceptibility model engine 210 using one or more machine learning models. Machine learning engine 230 may be trained using output from data validation engine 222 or performance measurement data from feedback engine 226 based on one or more outcome metrics 240. Machine learning engine 230 may be configured to predict a set of optimal dataset or data subset based on the training data. In some embodiments, machine learning engine 230 may be configured to predict an optimal adjusted data enrichment rate based on the training data. In some embodiments, an optimal dataset or data subset may comprise data that is associated with a set of susceptibility data generated by data generation engine 220 that has an optimal data validation score from data validation engine 222. For instance, in a cancer diagnosis setting, machine learning engine 230 may determine that a subset of multi-domain patient data 201 (e.g., comprising of a specific combination of multi-domain data such as proteomic data, patient characteristic data, or environmental data), in combination with a specific data model, may be used to generate a set of susceptibility data that is associated with an optimal data validation score based on output from data validation engine 222.

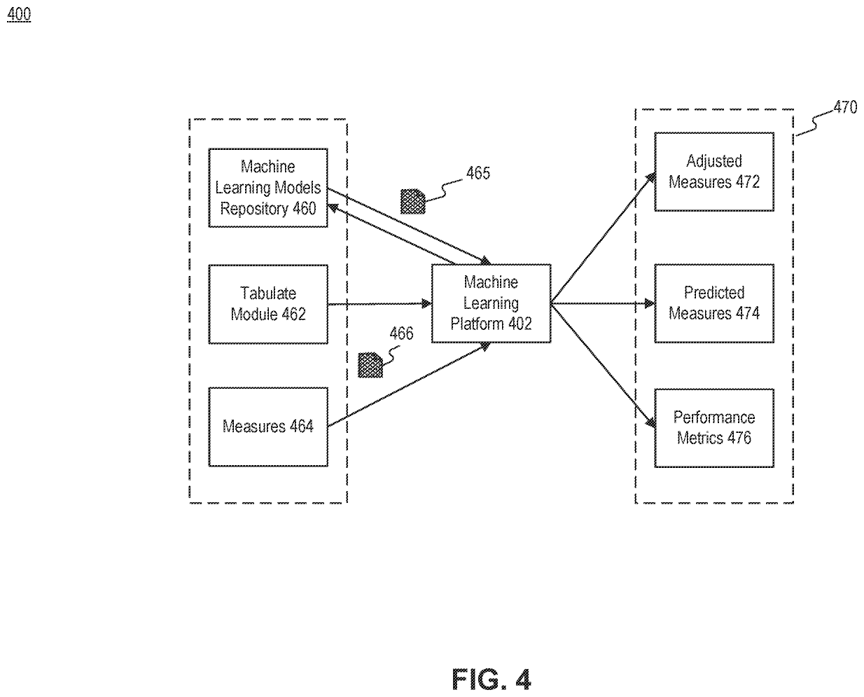

Machine learning engine 230 may measure the efficacy of the one or more outcomes based on one or more outcome metrics as exemplified by outcomes metrics 240. Machine learning engine 230 may measure an outcome efficacy based on output from data validation engine 222 or feedback engine 226. Machine learning engine 230 may perform iterative cycles of training and hypothesis refinement by automatically generating an alternative hypothesis based on a different subset of data within the multi-domain patient data 201, or a different data enrichment rate. In some embodiments, machine-learning engine 230 may also perform hypothesis refinement based on user-defined data selection or user-defined data enrichment rate as part of the input data from data input engine 110 in FIG. 1. Machine learning engine 230 may perform iterative cycles of hypothesis generation, validation, and refinement until an efficacy outcome measure, such as one from outcome metrics 240, reaches a threshold value. In some embodiments, machine learning engine 230 may be exemplified by machine learning platform 402 (shown in FIG. 4).

FIG. 3 is a block diagram illustrating an exemplary screening model engine for screening modulation and predictive output generation, validation, and refinement, according to some embodiments of the present disclosure.

Screening model engine 310 may include screening method selector engine 302, screening frequency modulation engine 304, screening application engine 306, predictive output generation engine 308, output validation engine 309, and screening refinement engine 312. In some embodiments, screening model engine 310 may be exemplified by screening model engine 134 as shown in FIG. 1.

Screening method selector engine 302 may receive as input a set of susceptibility data 301. In some embodiments, susceptibility data 301 may be generated by a susceptibility model engine as exemplified by susceptibility model engine 210 in FIG. 2. In some embodiments, susceptibility data 301 may be exemplified by output susceptibility data 260 in FIG. 2. In some embodiments, multi-domain patient data 201 shown in FIG. 2 can also be inputted into screening model engine 310. Screening method selector engine 302 may select one or more screening methods from a database of screening methods. In some embodiments, the database of screening methods may be exemplified by data storage 115 or data sources 101-104 as shown in FIG. 1. For instance, in the cancer diagnosis setting, the data base of screening methods may comprise screening methods corresponding to a particular type of cancer (e.g., mammography for breast cancer or colonoscopy for colon cancer). Screening method selector engine 302 may select one or more screening methods based on the susceptibility data 301. In some embodiments, screening method selector engine 302 may select screening method(s) using one or more machine learning algorithms based on machine learning engine 330. Screening method selector engine 302 may transmit the selected screening method(s) to screening frequency modulation engine 304.

Screening frequency modulation engine 304 may receive one or more screening methods from screening method selector engine 302. Screening frequency modulation engine 304 may set or adjust the frequency of one or more screening methods based on susceptibility data 301. For instance, in the cancer screening setting, screening frequency modulation engine 304 may set a high-frequency interval screening for a patient with high susceptibility to a specific cancer type, or a low-frequency interval screening for a patient with low susceptibility. Screening frequency modulation engine 304 may also set a screening frequency based on a cost-effective analysis of the screening method. Screening frequency modulation engine 304 may also set a screening frequency based on an estimated harm analysis of applying the screening method to an individual patient. Screening frequency modulation engine 304 may also set a screening frequency based on a screening guideline from an external data source, as exemplified by data storage 115 or data sources 101-104 in FIG. 1. Screening frequency modulation engine 304 may transmit the selected screening method(s) with associated screening frequencies to screening application engine 306.

Screening application engine 306 may execute an application of one or more selected screening methods based on the associated screening frequency, as determined by screening frequency modulation engine 304. Screening application model 306 may generate a personalized screening model based on the one or more screening methods, the associated screening frequencies, or susceptibility data 301. In some embodiments, such as in the cancer screening setting, screening application engine 306 may simulate the application of a screening method to an individual patient at a time interval based on the personalized screening model. In some embodiments, screening application engine 306 may simulate the application of a screening method based on the multi-domain patient data 201 or the susceptibility data 301 generated by susceptibility model engine 210. In some embodiments, screening application engine 306 may output a set of personalized application data based on the personalized screening model. In some embodiments, the set of personalized application data may comprise results associated with the application of the screening method. For instance, if the selected screening method is mammography, the application data may comprise a BI-RADS score or a detailed description of mammographic findings. The set of application data may also comprise periodic or progressive data associated with the screening method over multiple time intervals according to the screening frequency. For instance, if the screening frequency for a mammography screening method is set to be annual (i.e. on a yearly basis), then the set of application data may comprise a data series of BI-RADS scores or other successive mammographic findings over a predefined number of years for the individual patient. In some embodiments, the set of personalized application data may also comprise cost-effectiveness data associated with the screening method at a screening frequency over a pre-defined period of time. In some embodiments, cost-effectiveness data may comprise insurance data or healthcare coverage data associated with the individual patient. In some embodiments, the set of personalized application data may also comprise estimate risk data associated with any adverse events or harm from applying the screening method to the individual patient. Screening application engine 306 may transmit the set of personalized application data or the personalized screening model to predictive output generation engine 308.

Predictive output generation engine 308 may perform data analysis based on the set of personalized application data or personalized screening model from screening application engine 306. For instance, in the cancer screening and diagnosis setting, predictive output generation engine 308 may perform data analysis based on the personalized application data associated with one or more cancer screening methods administered at a certain screening frequency within the personalized screening model. Predictive output generation engine 308 may generate a set of predictive output data associated with a diagnosis of one or more types of cancers. In some embodiments, the set of predictive output data may comprise binary values (e.g., a positive cancer diagnosis vs. a negative cancer diagnosis). In some embodiments, the set of predictive outputs may comprise numerical scale of likelihood (e.g., on a scale of 1-10, the likelihood of having a specific type of cancer is 6 for an individual patient). In some embodiments, the set of predictive outputs may comprise categories of risk stratification (e.g., for an individual patient, there is a high, intermediate, or low risk for a specific cancer type). In some embodiments, the set of predictive output data may comprise data associated with a risk for a specific cancer type within a predetermined time frame from acquiring the patient data (e.g., within a specific number of years from the biological sampling of the patient).

Screening model engine 310 may include output validation engine 309. Output validation engine 309 may receive a set of predictive output data generated by predictive output generation engine 308. In some embodiments, output validation engine 309 may perform outputting of the set of predictive output data, as exemplified by output data 360. In some embodiments, output data 360 may be stored in a database, as exemplified by data storage 115 or data sources 101-104 as shown in FIG. 1. Output validation engine 309 may perform validation of the generated predictive output data by interacting with feedback engine 320. In some embodiments, feedback engine 320 may be exemplified by feedback engine 140 in FIG. 1. In some embodiments, feedback engine 320 may apply the personalized screening model from screening application engine 306 to a wider set of data to determine the accuracy of the model. In some embodiments, feedback engine 320 may utilize one or more outcome metrics as in outcome metrics 340 and generate a set of performance measurement data based on the generated predictive output data or the personalized screening model. In some embodiments, outcome metrics 340 may comprise a positive predictive value, a screening burden measurement, or an estimated risk measurement. For instance, in the cancer diagnosis setting, outcome metrics 340 may comprise a positive predictive value as an indication of the rate of accurate predictive output of cancer diagnosis by predictive output generation engine 308 in screening model engine 310. A screening burden measurement may be a measure based on a cost-effectiveness analysis based on the personalized screening model (e.g., by applying a cancer screening method at a specific screening frequency by screening application engine 306). A screening burden measurement may be based on a set of cost-effectiveness data. In some embodiments, the set of cost-effectiveness data may be based on the patient's insurance data or healthcare coverage data. An estimated risk measurement may be a measure based on any reported harm (e.g., from invasive screening techniques such as needle biopsies) to a patient due to the application of a screening method at a specific screening frequency per the personalized screening model. In some embodiments, the set of performance measurement data may comprise a numerical analysis based on a positive predictive value from outcome metrics 340. In some embodiments, the set of performance measurement data may comprise a numerical cost-effectiveness analysis of applying a screening method (e.g., a cancer screening method in the cancer diagnosis setting) at a given screening frequency. In some embodiments, the set of performance measurement data may comprise an estimated risk analysis based on reported harm (e.g., from invasive screening techniques to a patient in the cancer diagnosis setting) due to the application of the screening method at a given screening frequency.

Feedback engine 320 may transmit the performance measurement data to output validation engine 309. Output validation engine 309 may generate a screening validation score for screening refinement based on the performance measurement data. In some embodiments, the screening validation score for screening refinement may comprise a binary value (e.g., “screening refinement needed”, “screening refinement not needed”) or a gradient of values on a numerical scale (e.g., on a scale of 1-10, the need for screening refinement with regard to a specific data set is a “6 out of 10”). Output validation engine 222 may transmit the screening validation score for screening refinement to screening refinement engine 224.

Screening model engine 310 may include screening refinement engine 312. Screening refinement engine 312 may receive a screening validation score for screening refinement from output validation engine 309 or performance measurement data from feedback engine 320. Screening refinement engine 312 may perform screening refinement based on input from output validation engine 309 or a performance measurement data from feedback engine 320. Screening refinement engine 312 may perform screening refinement by generating an alternative screening method from a database of screening methods. Screening refinement engine 312 may perform screening refinement by modulating the screening frequency associated with a screening method. Screening refinement engine 312 may perform refinement based on the performance measurement data. Screening refinement engine 312 may interact with screening method selector engine 302 for iterative data refinement.

In some embodiments, screening model engine 310 may enable iterative cycles of screening refinement. An iterative cycle of screening refinement may comprise screening refinement engine 312 transmitting an alternative screening method or a modulated screening frequency to screening method selector engine 302. An iterative cycle may comprise screening method selector engine 302 selecting the alternative screening method based on the output from screening refinement engine 312. For instance, in breast cancer screening, screening refinement engine 312 may generate an alternative method to mammography, such as breast ultrasound or breast MRI scan. An iterative cycle of screening refinement may also comprise the screening frequency modulation engine 304 adjusting a screening frequency associated with an alternative screening method as selected by screening method selector engine 302. For instance, screening frequency modulation engine 304 may increase or decrease the time interval between screening based on a modulated frequency from screening refinement engine 312. An iterative cycle of screening refinement may also comprise screening application engine 306 generating a refined screening model based on the one or more alternative screening methods, the modulated screening frequencies, or susceptibility data 301. An iterative cycle of screening refinement may also comprise predictive output generation engine 308 generating a set of refined predictive output data based on the refined screening model. An iterative cycle of screening refinement may also comprise output validation engine 309 interacting with feedback engine 320 to validate the refined predictive output data. In some embodiments, iterative cycles of screening refinement may continue until output validation engine 309 or feedback engine 320 determines that one or more outcome metrics 340 has achieved a threshold value. In some embodiments, output validation engine 309 may perform outputting of the set of refined predictive output data, as exemplified by output data 360. In some embodiments, output data 360 may be stored in a database, as exemplified by data storage 115 or data sources 101-104 as shown in FIG. 1.

Screening model engine 310 may interact with machine learning engine 330. According to some embodiments, machine learning engine 330 may analyze one or more dataset or data subsets utilized by screening model engine 210 using one or more machine learning models. Machine learning engine 330 may be trained using output from output validation engine 309 or performance measurement data from feedback engine 320 based on one or more outcome metrics 340. Machine learning engine 330 may be configured to predict a set of optimal predictive output data based on a training data set. For instance, in the cancer screening and diagnosis setting, machine learning engine 330 may be configured to produce cancer diagnoses optimized for precision or accuracy. In some embodiments, machine learning engine 330 may be configured to predict an optimal screening method or an optimal screening frequency based on a training data set. In some embodiments, the training data set may comprise data from susceptibility data 301. In some embodiments, the training data set may comprise from external data sources such as in data storage 115 or data sources 101-104 as in FIG. 1. In some embodiments, an optimal screening method or optimal screening frequency may be associated with an optimal screening validation score from output validation engine 309. For instance, in a breast cancer diagnosis setting, machine learning engine 330 may determine that, based on output from output validation engine 309 or feedback engine 320, the optimal screening method for an individual patient with a specific susceptibility profile based on the susceptibility data 301 (e.g., age over 50, positive family history of breast cancer, negative smoking history, etc.) is mammography at a modulated screening frequency of once per year, based on data from screening refinement engine 312.

Machine learning engine 330 may measure the efficacy of the one or more outcomes based on one or more outcome metrics as exemplified by outcomes metrics 340. Machine learning engine 330 may measure an outcome efficacy based on output from output validation engine 309 or feedback engine 320. Machine learning engine 330 may perform iterative cycles of training and hypothesis refinement by automatically generating an alternative hypothesis based on a different subset of data within the susceptibility data 301, a different screening method, or screening frequency. In some embodiments, machine-learning engine 330 may also perform hypothesis refinement based on user-defined data selection or user-defined screening method or screening frequency as part of the input data from data input engine 110 in FIG. 1. Machine learning engine 330 may perform iterative cycles of hypothesis generation, validation, and refinement until an efficacy outcome measure, such as one from outcome metrics 340, reaches a threshold value. In some embodiments, machine learning engine 330 may be exemplified by machine learning platform 402 (shown in FIG. 4).

FIG. 4 is a block diagram illustrating various exemplary components of a machine learning platform, according to some embodiments of the present disclosure.

As illustrated in FIG. 4, Machine Learning (ML) platform 402 may use Machine Learning (ML) models in Machine Learning (ML) models repository 460 as input to generate performance measures. In some embodiments, machine learning platform 402 may be exemplified by machine learning engine 230 as shown in FIG. 2. In some embodiments, machine learning platform 402 may be exemplified by machine learning engine 330 as shown in FIG. 3. Performance measures 470 generated by ML platform 402 may include adjusted measures 472, predicted measures 474, and performance metrics 476.

ML platform 402 may generate performance measures by taking additional

measures generated by tabulate module 462 as input. In some embodiments, ML platform 402 may also take additional measures from measures 464. In some embodiments, measures 464 are stored in an external data source as input. In some embodiments, tabulate module 462 may directly supply measures generated by querying a database.

ML platform 402 may use input measure 466 from measures 464 and ML model 465 from ML models repository 460 to generate performance measures 470, including adjusted measures 472, predicted measures 474, performance metrics 476. Adjusted measures 472 may be adjustments to input measure 466 adjusted for the susceptibility of a patient for a specific cancer type based on susceptibility data generated by a susceptibility model (as shown in FIG. 2). ML platform 402 may make multiple adjustments to various data parameters associated with input measure 466. In some embodiments, ML platform 402 may also generate multiple adjusted measures for each data type or data source associated with input measure. ML platform 402 may predict the performance of ML models used by susceptibility model engine 210 (as shown in FIG. 2) in generating susceptibility data. ML platform 402 may predict the performance of ML models used by screening model engine 310 (as shown in FIG. 3) in modulating screening or predicting output.

ML platform 402 may use different layers of ML model 465 to generate different types of measures in performance measures 470. In some embodiments, ML platform 402 may use different ML models for each type of performance measure. In some embodiments, ML platform 402 may generate ML models as part of performance measures generation. ML platform 402 may store the generated ML models in ML models repository 460. ML platform 402 may generate new ML models by adjusting ML model 465 based on the generated performance measures 470.

ML platform 402 may link the performance measures 470 with input measure 466. The relationships between the adjusted measures 472 or predicted measures 474 and input

measure 466 may be stored in an external data storage. In some embodiments, ML platform 402 may also store relationships between input measure 466 and performance metrics 476 of a machine learning model of ML models repository 460. The performance metrics 476 may indicate the variance between predictive outputs of ML models used by screening model engine 310 and the measures of the outcomes as exemplified by feedback engine 320 as in FIG. 3. The relationship between performance metrics 476 and input measure 466 may only exist when the variance is beyond a threshold. ML Platform 402 may request the measurement system 100 to store the performance measures 470 in a data storage.

A ML model in ML models repository 460 may be based on one or more ML algorithms. In some embodiments, the ML algorithms may include, for example, Viterbi algorithms, Naïve Bayes algorithms, neural networks, elastic net regression models etc. or joint dimensionality reduction techniques (e.g., cluster canonical correlation analysis, partial least squares, bilinear models, cross-modal factor analysis). In some embodiments, the ML model may include accelerated failure time models or proportional hazards models. In some embodiments, the ML models may be based on a Weibull distribution, a log-logistic distribution, an exponential distribution, or a gamma distribution. The type of model selected from model repository 460 may impact the inputs that are needed for both susceptibility model engine 210 and screening model engine 310.

In some embodiments, the at least one ML model 465 may also be trained, for example, using a supervised learning method (e.g., gradient descent or stochastic gradient descent optimization methods). In some embodiments, the ML models may be trained based on user-generated training data or automatically generated markup data.

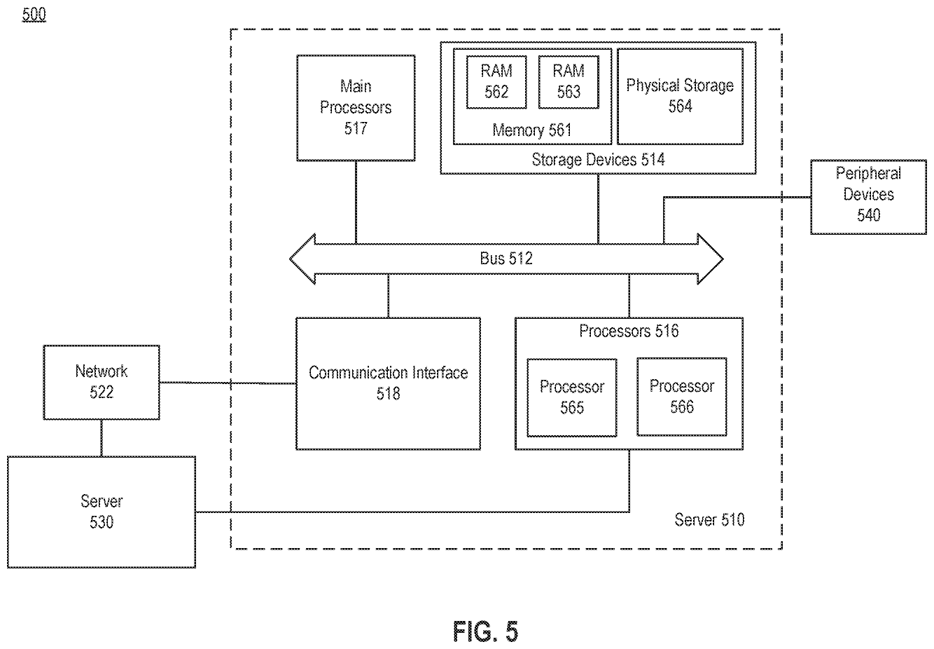

FIG. 5 illustrates a schematic diagram of an exemplary server of a distributed system, according to some embodiments of the present disclosure. In some embodiments, system 100 and its components (as shown in FIG. 1) may be implemented in a distributed computing system as exemplified by distributed computing system 500. According to FIG. 5, server 510 of distributed computing system 500 comprises a bus 512 or other communication mechanisms for communicating information, one or more processors 516 communicatively coupled with bus 512 for processing information, and one or more main processors 517 communicatively coupled with bus 512 for processing information. Processors 516 can be, for example, one or more microprocessors. In some embodiments, one or more processors 516 comprises processor 565 and processor 566, and processor 565 and processor 566 are connected via an inter-chip interconnect of an interconnect topology. Main processors 517 can be, for example, central processing units (“CPUs”).

Server 510 further comprises storage devices 514, which may include memory 561 and physical storage 564 (e.g., hard drive, solid-state drive, etc.). Memory 561 may include random access memory (RAM) 562 and read-only memory (ROM) 563. Storage devices 514 can be communicatively coupled with processors 516 and main processors 517 via bus 512. Storage devices 514 may include a main memory, which can be used for storing temporary variables or other intermediate information during execution of instructions to be executed by processors 516 and main processors 517. Such instructions, after being stored in non-transitory storage media accessible to processors 516 and main processors 517, render server 510 into a special-purpose machine that is customized to perform operations specified in the instructions. The term “non-transitory media” as used herein refers to any non-transitory media storing data or instructions that cause a machine to operate in a specific fashion. Such non-transitory media can comprise non-volatile media or volatile media. Non-transitory media include, for example, optical or magnetic disks, dynamic memory, a floppy disk, a flexible disk, hard disk, solid state drive, magnetic tape, or any other magnetic data storage medium, a CD-ROM, any other optical data storage medium, any physical medium with patterns of holes, a RAM, a PROM, and an EPROM, a FLASH-EPROM, NVRAM, flash memory, register, cache, any other memory chip or cartridge, and networked versions of the same.

Server 510 can transmit data to or communicate with another server 530 through a network 522. Network 522 can be a local network, an internet service provider, Internet, or any combination thereof. Communication interface 518 of server 510 is connected to network 522, which can enable communication with server 430. In addition, server 510 can be coupled via bus 512 to peripheral devices 540, which comprises displays (e.g., cathode ray tube (CRT), liquid crystal display (LCD), touch screen, etc.) and input devices (e.g., keyboard, mouse, soft keypad, etc.).

Server 510 can be implemented using customized hard-wired logic, one or more ASICs or FPGAs, firmware, or program logic that in combination with the server causes server 510 to be a special-purpose machine.

Various forms of media can be involved in carrying one or more sequences of one or more instructions to processors 516 or main processors 517 for execution. For example, the instructions can initially be carried out on a magnetic disk or solid-state drive of a remote computer. The remote computer can load the instructions into its dynamic memory and send the instructions over a telephone line using a modem. A modem local to server 510 can receive the data on the telephone line and use an infra-red transmitter to convert the data to an infra-red signal. An infra-red detector can receive the data carried in the infra-red signal, and appropriate circuitry can place the data on bus 512. Bus 512 carries the data to the main memory within storage devices 514, from which processors 516 or main processors 517 retrieves and executes the instructions.

Multi-domain Data-driven System 100 or one or more of its components may reside on either server 510 or 530 and may be executed by processors 516 or 517. In some embodiments, the components of system 100 may be spread across multiple servers 510 and 530. For example, Data Input Engine 110 or Featurization Engine 120 may be executed on multiple servers. Similarly, Analysis Engine 130 or Feedback Engine 140 may be maintained by multiple servers 510 and 530.

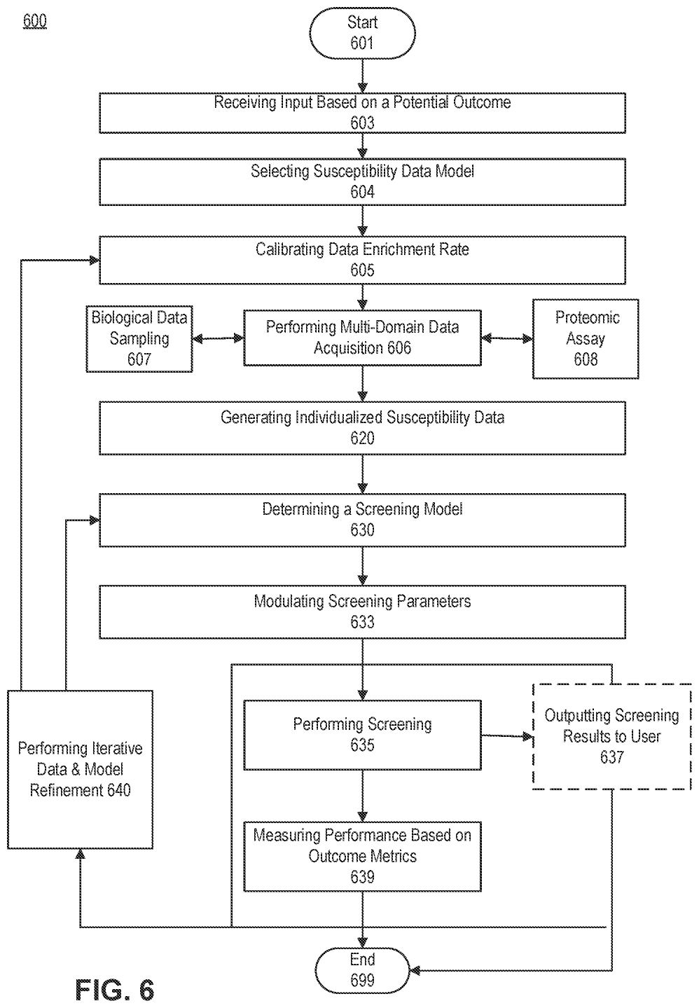

FIG. 6 is a flow diagram illustrating an exemplary process for receiving input based on a potential outcome, performing multi-domain data acquisition, generating individualized data, performing screening and refinement of the data and model based on measured performance, according to some embodiments of the present disclosure.

Process 600 can be performed by a system, such as system 100 of FIG. 1. In some embodiments, process 600 can be implemented using one or more instructions that can be stored on a computer readable medium (e.g., storage device 514 of FIG. 5).

In some embodiments, process 600 begins at step 603. In step 603, the system may receive data input across multiple data sources or data domains based on a potential outcome. For instance, in the cancer diagnosis or screening setting, input data may comprise data associated with a cancer type, a cancer prevalence, a cancer prognosis, timing of cancer diagnosis, or a cancer stage, while a potential outcome may comprise a diagnosis of a specific cancer type. In some embodiments, input data may comprise proteomic data, patient characteristics data (e.g., age, gender, race/ethnicity, height/weight of a patient), demographical data, environmental data (e.g., smoking history, diet type, etc.), data obtained from a biological sampling (e.g., relating to a sample of blood, plasma, serum, or urine), medical history data, clinical visit data, surgical history data, family history data, genetic data, or immunological data. In some embodiments, input data my comprise cost-effectiveness data, comprising insurance data or healthcare coverage data.

In step 604, using the input data, the system may select a susceptibility data model used for determining a user's susceptibility with regard to a potential outcome. For instance, in a cancer diagnosis setting, the system may select a data model from a plurality of data models stored within a model databank (as exemplified by 108 of FIG. 1) that is used to determine a patient's individual susceptibility to a specific type of cancer. In some embodiments, the system may select a data model based on a subset of the input data. For instance, the system may select a model based on the subset of the input data comprising cancer type, prevalence, stage, or severity. In some embodiments, the system may select a data model to compensate for certain characteristics in the targeted outcome. For instance, the system may select different data models for cancers of lower prevalence than for commonly occurring cancer types. In some embodiments, some of the input data may be more or less relevant based on which susceptibility data model is selected. For instance, a patient's smoking history may be more relevant with respect to lung cancer when compared to colon cancer. Accordingly, less relevant input data may be discarded.

In step 605, the system may select or calibrate a value comprising a data enrichment rate from the input data. In some embodiments, the data enrichment rate value may comprise a coefficient (e.g., 2×, 5×, 10×, etc.) or a percentage (e.g., 10%, 20%, etc.). The system may enrich the input data or data obtained via multi-domain data acquisition (e.g., as in step 606) based on the data enrichment rate. For instance, based on a data enrichment rate of 10%, the system may increase the amount of data that it receives or acquires accordingly by 10%.

In step 606, the system may perform data acquisition from multiple data domains or data sources based on the data parameters of the selected data model. The system may enrich the acquired data based on a data enrichment rate as selected in step 605. In some embodiments, the system may perform data acquisition by performing biological data sampling 607, in which a biological sample relating to a sample of blood, plasma, serum, or urine is obtained. In some embodiments, the system may perform data acquisition via a proteomic assay 608 based on an obtained biological sample.

In step 620, the system may generate a set of susceptibility data for an individual user relating to a potential outcome. In some embodiments, the set of susceptibility data may be generated by susceptibility model engine 210 as shown in FIG. 2. In some embodiments, for instance in the cancer diagnosis setting, the system may generate a set of susceptibility data for a specific patient relating to a specific cancer type diagnosis based on the input data or the acquired data, and the selected susceptibility data model. The generated susceptibility data may comprise an individualized susceptibility score based on susceptibility estimation analysis. In some embodiments, a susceptibility score may comprise a likelihood percentage for the occurrence of the potential outcome or a category of risk stratification (e.g., in cancer diagnosis). In some embodiments, the susceptibility data or susceptibility score may comprise a multiplicative coefficient representing a likelihood of occurrence of a potential outcome for an individual (e.g., a patient) relative to a reference population. For instance, a patient with a specific set of susceptibilities may have 2× or 10× greater risk of developing a cancer comparing to the general population. In some embodiments, the susceptibility data may comprise data associated with the likelihood of occurrence of a potential outcome within a predetermined time frame. For instance, in the cancer diagnosis setting, the susceptibility data may comprise a patient's susceptibility score to a specific type of cancer over a particular time frame (e.g., from the time when the patient's biological sampling data was obtained).

In step 630, the system may determine a screening model for the potential outcome of interest. In some embodiments, the system may determine a screening model based on individualized susceptibility data associated with the potential outcome of interest. In some embodiments, the screening model may comprise one or more screening methods, each of which is associated with a screening frequency.

In step 633, the system may modulate the screening parameters associated with a screening model or screening method. In some embodiments, the system may select among various screening methods. In some embodiments, the system may modulate the frequency of screening for one or more screening methods. In some embodiments, the screening method may be selected by screening model engine 310 as shown in FIG. 3. In some embodiments, modulating the frequency of the screening method may be performed by screening model engine 310. In some embodiments, for instance in the cancer screening setting, the system may modulate a screening frequency by setting a higher-frequency interval screening for a patient with high susceptibility to a specific cancer type, or a lower-frequency interval screening for a patient with low susceptibility. The system may also modulate the screening frequency based on a cost-effective analysis of the screening method, or an estimate harm analysis of applying the screening method to an individual patient. The system may also modulate the screening frequency based on a screening guideline from an external data source.

Process 600 then moves to step 635. In step 635, the system may perform screening for the potential outcome of interest using a screening model. In some embodiments, such as in the cancer screening setting, the system may simulate the application of a screening method to an individual patient at a time interval based on the personalized screening model, or input data or acquired data. In some embodiments, the system may output results associated with the application of the screening. For instance, if the selected screening method is mammography, the system may output a BI-RADS score or a detailed description of mammographic findings. In some embodiments, the system may also output cost-effectiveness data associated with the screening method at a screening frequency over a pre-defined period of time. In some embodiments, cost-effectiveness data based on the patient's insurance data or healthcare coverage data. In some embodiments, the system may also output estimate risk data associated with adverse events or harm associated with application of the screening method.

Also in step 635, the system may generate predictive outputs associated with the potential outcome of interest based on the results associated with the application of the screening. For instance, in the cancer screening and diagnosis setting, the system may generate a set of predictive output data associated with a diagnosis of one or more types of cancers. In some embodiments, the set of predictive output data may comprise binary values (e.g., a positive cancer diagnosis vs. a negative cancer diagnosis). In some embodiments, the set of predictive outputs may comprise numerical scale of likelihood (e.g., on a scale of 1-10, the likelihood of having a specific type of cancer is 6 for an individual patient). In some embodiments, the set of predictive outputs may comprise categories of risk stratification (e.g., for an individual patient, there is a high/intermediate/low risk for a specific cancer type).

Also in step 635, the system may optionally output data associated with the screening results or generated predictive output data to a user or operator. In some embodiments, the system output data via an external display device as exemplified by 150 of FIG. 1.

In step 639, the system may measure the performance of the screening model using one or more outcome metrics. In some embodiments, outcome metrics may comprise a positive predictive value, a screening burden measurement, or an estimated risk measurement. In some embodiments, the system may determine a set of performance measurement data based on one or more outcome metrics. In some embodiments, the system may validate the screening model based on the performance measurement data.

In step 640, the system may make a determination for additional cycles of iterative data refinement or data model refinement based on validation of the screening model, the performance measurement data, or one or more outcome metrics. The system may perform data refinement by adjusting the data enrichment rate and generate a set of refined susceptibility data based on the adjusted data enrichment rate. The system may also perform screening refinement by selecting alternative screening methods and modulating the screening frequencies, and generate a refined screening model based on the alternative screening method and modulated screening frequencies. In some embodiments, the system may perform iterative cycles of data refinement or screening refinement until it reaches a determination that the measured performance associated with the potential outcome of interest, based on one or more outcome metrics, has achieved a threshold value.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Example embodiments are described above with reference to flowchart illustrations or block diagrams of methods, apparatus (systems) and computer program products. It will be understood that each block of the flowchart illustrations or block diagrams, and combinations of blocks in the flowchart illustrations or block diagrams, can be implemented by computer program product or instructions on a computer program product. These computer program instructions may be provided to a processor of a computer, or other programmable data processing apparatus to produce a machine, such that the instructions, which execute via the processor of the computer or other programmable data processing apparatus, create means for implementing the functions/acts specified in the flowchart or block diagram block or blocks.

These computer program instructions may also be stored in a computer readable medium that can direct one or more hardware processors of a computer, other programmable data processing apparatus, or other devices to function in a particular manner, such that the instructions stored in the computer readable medium form an article of manufacture including instructions that implement the function/act specified in the flowchart or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions that execute on the computer or other programmable apparatus provide processes for implementing the functions/acts specified in the flowchart or block diagram block or blocks.

Any combination of one or more computer readable medium(s) may be utilized. The computer readable medium may be a non-transitory computer readable storage medium. In the context of this document, a computer readable storage medium may be any tangible medium that can contain or store a program for use by or in connection with an instruction execution system, apparatus, or device.

Program code embodied on a computer readable medium may be transmitted using any appropriate medium, including but not limited to wireless, wireline, optical fiber cable, RF, IR, etc., or any suitable combination of the foregoing.

Computer program code for carrying out operations, for example, embodiments may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The flowchart and block diagrams in the figures illustrate examples of the architecture, functionality, and operation of possible implementations of systems, methods, and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts, or combinations of special purpose hardware and computer instructions.

It is understood that the described embodiments are not mutually exclusive, and elements, components, materials, or steps described in connection with one example embodiment may be combined with, or eliminated from, other embodiments in suitable ways to accomplish desired design objectives.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A non-transitory computer readable medium including instructions that are executable by one or more processors to cause a system to perform a method comprising:
receiving input data associated with one or more types of cancer and a patient;
determining a susceptibility model and a data enrichment rate based on the input data;
acquiring data associated with the patient from a plurality of data domains based on the susceptibility model and the data enrichment rate, wherein the patient data comprises at least proteomic data;
generating, using a machine learning algorithm, cancer susceptibility data associated with the patient using the susceptibility model and the patient data;
determining a screening model of the one or more types of cancer for the patient based on the cancer susceptibility data;
providing information for screening the patient for the one or more types of cancer based on the screening model and patient data.

2. The non-transitory computer readable medium of claim 1, wherein the input data comprises at least one of a cancer type, a cancer prevalence, a cancer diagnosis timing, a cancer prognosis, or a cancer stage.

3. The non-transitory computer readable medium of claim 1, wherein the patient data further comprises patient characteristics data, insurance data, healthcare coverage data, medical history data, genetic data, immunological data, environmental data, or biological sampling data from the patient.

4. The non-transitory computer readable medium of claim 1, wherein the proteomic data is based on a biological sample of the patient.

5. The non-transitory computer readable medium of claim 1, wherein the instructions that are executable by the one or more processors to cause the system to further perform:
determining, from the patient data, a set of features associated with the patient's susceptibility to the one or more types of cancer.

6. The non-transitory computer readable medium of claim 1, wherein determining a susceptibility model comprises selecting a susceptibility model from a plurality of data models within a model databank.

7. The non-transitory computer readable medium of claim 1, wherein the screening model for the patient comprises one or more screening methods, each associated with one or more screening schedules.

8. The non-transitory computer readable medium of claim 1, wherein the instructions that are executable by the one or more processors to cause the system to further perform:
iteratively refining the screening model using one or more machine learning algorithms, by adjusting a screening schedule of a screening method based on one or more outcome metrics, until the one or more outcome metrics reach a threshold value.

9. The non-transitory computer readable medium of claim 7, wherein the outcome metric comprises at least one of a positive predictive value, a screening burden measurement, or an estimated risk measurement.

10. The non-transitory computer readable medium of claim 7, wherein the instructions that are executable by the one or more processors to cause the system to further perform:
iteratively refining, until the one or more outcome metrics reach a threshold value, the susceptibility model using one or more machine learning algorithms by adjusting the data enrichment rate based on the one or more outcome metrics and generating a refined set of susceptibility data based on the adjusted enrichment rate.

11. A method for data modelling and analysis, comprising:
receiving input data associated with one or more types of cancer and a patient;
determining a susceptibility model and a data enrichment rate based on the input data;
acquiring data associated with the patient from a plurality of data domains based on the susceptibility model and the data enrichment rate, wherein the patient data comprises at least proteomic data;
generating, using one or more machine learning algorithms, a set of cancer susceptibility data associated with the patient based on the susceptibility model and the patient data;
determining, using one or more machine learning algorithms, a screening model of the one or more types of cancer for the patient based the cancer susceptibility data;
screening the patient for the one or more types of cancer based on the screening model.

12. The method of claim 11, wherein the input data comprises at least a cancer type, a cancer prevalence, a cancer diagnosis timing, a cancer prognosis, a cancer stage, or any combination thereof.

13. The method of claim 11, wherein the patient data further comprises patient characteristics data, medical history data, insurance data, healthcare coverage data, genetic data, immunological data, environmental data, or biological sampling data from the patient.

14. The method of claim 11, wherein the proteomic data is based on a biological sample of the patient.

15. The method of claim 11, wherein determining a susceptibility model comprises selecting a susceptibility model from a plurality of data models within a model databank.

16. The method of claim 11, further comprising:
determining, from the patient data, a set of features associated with the patient's susceptibility to the one or more types of cancer.

17. The method of claim 11, wherein the screening model for the patient comprises one or more screening methods, each associated with one or more screening schedules.

18. The method of claim 11, further comprising:
iteratively refining, until the one or more outcome metrics reach a threshold value, the screening model using one or more machine learning algorithms by adjusting a screening schedule of a screening method based on one or more outcome metrics.

19. The method of claim 18, wherein the outcome metric comprises a positive predictive value, a screening burden measurement, an estimated risk measurement, or any combination thereof.

20. The method of claim 18, further comprising:
iteratively refining the susceptibility model using one or more machine learning algorithms by adjusting the data enrichment rate based on the one or more outcome metrics and generating a refined set of susceptibility data based on the adjusted enrichment rate.

21. A computer-implemented system for data modelling and analysis, the system comprising:
a memory storing instructions; and
at least one processor configured to execute the instructions to cause the computer-implemented system to perform operations comprising:
receiving input data associated with one or more types of cancer and a patient;
determining a susceptibility model and a data enrichment rate based on the input data;
acquiring data associated with the patient from a plurality of data domains based on the susceptibility model and the data enrichment rate, wherein
the patient data comprises at least proteomic data;
generating, using a machine learning algorithm, cancer susceptibility data associated with the patient using the susceptibility model and the patient data;
determining a screening model of the one or more types of cancer for the patient based on the cancer susceptibility data;
providing information for screening the patient for the one or more types of cancer based on the screening model and patient data.
